(19) United States
(12) Patent Application Publication
(10) Pub. No.: US 2022/0357819 A1
(43) Pub. Date: Nov. 10, 2022

(54) METHOD AND SYSTEM FOR BUILDING A FAMILY TREE

(71) Applicant: Andrey Kozorezov, Bothell, WA (US)

(72) Inventor: Andrey Kozorezov, Bothell, WA (US)

(21) Appl. No.: 17/856,963

(22) Filed: Jul. 2, 2022

Related U.S. Application Data
(60) Provisional application No. 63/183,181, filed on May 3, 2021.

(51) Int. Cl. G06F 3/0481; G06F 16/957; H04L 9/40; G06T 11/20

(52) U.S. Cl. CPC G06F 3/0481; G06F 16/9577; H04L 63/083; G06T 11/206; G06Q 50/01

(57) ABSTRACT

The embodiments herein provide a method and system for building a family tree through a social network. The method includes receiving login credentials from a first person, providing access to a profile of the first person in the social network based on the login credentials, receiving a first triggering event from the first person; wherein the first triggering event occurs when the first person actuates first web element, providing a user interface for building a family tree on receiving the first triggering event from the first person, receiving a second triggering event from the first person; wherein the second triggering event occurs when the first person actuates second web element in the profile of the first person, saving the changes related to the family tree on receiving the second triggering event from the first person, and displaying the family tree on the user interface of the social network.

### CROSS-REFERENCE TO RELATED PATENT DOCUMENTS

This patent application claims the benefit of priority of U.S. Provisional Application No. 63/183,181, entitled “SYSTEM AND METHOD PROVIDING A SOCIAL NETWORK PLATFORM FOR BUILDING A FAMILY TREE,” filed 9 Jul. 2021, which is hereby incorporated herein by reference in its entirety.

### TECHNICAL FIELD

The present invention relates generally to a social network, and more particularly, to a method and system for building a family tree through the social network.

### BACKGROUND

A family tree, also called a genealogy, is a visual representation that represents family relationships in a conventional tree structure. Further, the family tree assists to record the relations of relatives and the degrees of the relatives (such as Grandfather is the first order relative and father and uncle are second order relatives, etc.). Previously, in many traditional families, the family trees are recorded and used for inviting all the members of the family to family functions. However currently, members of the family are spread in many geographical areas, and they are disconnected, still the family trees must be used in many fields, such as connection of relatives, inheritance of estate, marriages, etc. Thus, the family trees have their usages.

Currently, when an individual wish to create his/her family tree he/she will often talk to as many relatives as he/she can contact and get their best recollections of the family relationships. From this information, the individual will construct the best possible family tree from the available information. However, a family tree constructed in this manual manner may often be incomplete since it relies on the memories of those currently living. Further, the individual and his/her known relatives may not be aware of all the events in their immediate family, or extended family which is required for family tree construction.

There are various systems, methods, software applications, and website exist to build the family tree such as U.S. Pat. No. 8,224,862B2 issued to Sacks discloses a system and method for a networked viral family tree. The method comprises receiving the first profile from a first client. The first profile includes a first e-mail address. A second e-mail address is received from the first client. The second e-mail address is associated with a second profile. The second profile is created dynamically when the client provides the second e-mail address. An invitation is sent to the second e-mail address to provide additional profile information associated with the second profile. One or more relationships are associated with the first profile and the second profile to generate a family tree. One or more user interfaces are provided through which the first client provides a plurality of e-mail addresses corresponding to members of the family tree and are associated with a plurality of profiles.

US publication US20050015710A1 filed by Williams, Lori Beth discloses a family website system having a group webpage for displaying in a visually intuitive manner to various sub-group webpages of the family website system. Several sub-group webpages may include more detailed information relating to a particular family unit and/or to provide specific services and information such as a calendar of family events, a message center, a family photograph album, medical statistics, words of wisdom, personal resumes, and so on. Each member may create and display a personal webpage having multimedia information in a design and layout that fits the characteristics of the member. Any changes made to the various pages of the website may be indicated in a notification section on relevant web pages on the website, such as on a section of the group webpage. Each family member may create a personal homepage that is customizable to contain selected features of the family website system.

Currently, none of the existing systems and methods can remind about the key events such as birthday to the individual if someone of his/her family member is not among his/her friend's list on a social network platform he/she currently uses. Further, the existing prior arts and the family tree builder application or websites do not encourage and help socializing and to keep in touch.

Although family tree building methods and systems are proposed in the past, they have their own shortcomings or limitations, thus there still exist a need for a more reliable solution which can provide a simple, effective, and inexpensive way to gather information to construct a family tree and to broadly distribute the final result. Accordingly, proposed is a method and system for building a family tree though the social network.

### SUMMARY

It will be understood that this disclosure in not limited to the particular systems, and methodologies described, as there can be multiple possible embodiments of the present disclosure which are not expressly illustrated in the present disclosure. It is also to be understood that the terminology used in the description is for the purpose of describing the particular versions or embodiments only, and is not intended to limit the scope of the present disclosure.

It is an objective of the present invention to provide a method and system for building a family tree though a social network.

It is another objective of the present invention to merge old school family tree, genealogical record keeping, contacts information rolodex, photo album, calendar and planner, geographical map of the world and new social sites technologies as well as Internet and mobile devices phone for building a family tree though a social network.

It is another objective of the present invention to allow family members participate in building their family tree.

It is another objective of the present invention to accepts an invitation from a second person to add him/her to the family tree and verify the second person based on data related to the invitation.

In an embodiment, the present invention provides system and method for building a family tree though a social network. The method includes receiving login credentials from a first person, providing access to a profile of the first person in the social network based on the login credentials, receiving a first triggering event from the first person; wherein the first triggering event occurs when the first person actuates first web element in the profile of the first person, providing a user interface for building a family tree on receiving the first triggering event from the first person, receiving a second triggering event from the first person; wherein the second triggering event occurs when the first person actuates second web element in the profile of the first person, saving the changes related to the family tree on receiving the second triggering event from the first person, determining whether a third triggering event from the first person is received; wherein the third triggering event occurs when the first person accepts an invitation from a second person, and displaying the family tree on the user interface of the social network based on the determination of receiving the third triggering event from the first person.

In an embodiment, displaying the family tree further includes displaying the family tree on the user interface of the social network if it is determined that no third triggering event from the first person is received, and displaying an updated family tree on the user interface of the social network if it is determined that the third triggering event from the first person is received.

In an embodiment, the displaying of the updated family tree further includes updating the family tree automatically based on the third triggering event if it is determined that the third triggering event from the first person is received and displaying the updated family tree on the user interface of the social network.

In an embodiment, the family tree is created by the linkage of a node to one or more additional nodes, each linkage having one or more than one defined linkage type.

In an embodiment, the linkage types include one of familial relationship types, biological relationship types, and genetic relationship types.

In an embodiment, each node is associated with information pertaining to the individual that it represents and displays one or more nodes at a generation level.

In an embodiment, each of the nodes provides an indication related to the current living status of a person; wherein the current living status includes alive status or dead status. Further, each node provides indication related to the active user and inactive user; wherein an active user represents an individual who has registered with the social network and an inactive user represents an individual who has not registered with the social network.

In an embodiment, a web element includes one of a widget, a graphical control element, and a graphical user interface element.

In an embodiment, the system provides a search interface for filtering data based on one or more criteria; wherein one or more criteria comprise age, gender, geographical location, distance, current living status, date, time period, and the like.

In an embodiment, the system provides web elements for zooming out and zooming in the family tree diagram.

In an embodiment, the social network includes one or more of instant messaging, electronic mail, blogging, photo albums, calendar, and online diaries.

In an embodiment, notifying users of the social network when information related to the user matches with the information of the first user; wherein the information related to the user comprises relative names, birthdays, birthplaces, and contact information.

In an embodiment, the system allows users of the social network to send requests to each other to collaborate and join family trees.

In an embodiment, accepting an invitation from a second person further comprising verifying the second person based on data related to the invitation.

These and other features and advantages of the present invention will become apparent from the detailed description below, in light of the accompanying drawings.

### BRIEF DESCRIPTION OF THE DRAWINGS

FIG. 1 illustrates a computing environment or general implementation for building a family tree through a social network, according to an exemplary embodiment of the present invention;

FIG. 2 illustrates an example login interface for the social network, according to an embodiment of the present invention;

FIG. 3A illustrates an example user interface for entering data related to a first person, according to an embodiment of the present invention;

FIG. 3B illustrates an example user interface of a user profile, according to an embodiment of the present invention;

FIG. 3C illustrates an example user interface for applying settings for the user profile, according to an embodiment of the present invention;

FIG. 4A illustrates an example search interface for filtering data based on one or more criteria, according to an embodiment of the present invention;

FIG. 4B illustrates an example user interface for displaying the filtered data based on one or more criteria, according to an embodiment of the present invention;

FIG. 5 illustrates an example user interface for creating an event, according to an embodiment of the present invention;

FIG. 6A illustrates an example user interface for displaying zoom-in of the family tree diagram, according to an embodiment of the present invention;

FIG. 6B illustrates an example user interface for displaying zoom out of the family tree diagram, according to an embodiment of the present invention;

FIG. 7 illustrates a flow diagram of a method for building the family tree through the social network, according to an exemplary embodiment of the present invention; and

FIG. 8 illustrates a system for building the family tree through the social network, according to an exemplary embodiment of the present invention.

### DETAILED DESCRIPTION

As used in the specification, the singular forms “a”, “an”, and “the” may also include plural references. For example, the term “an article” may include a plurality of articles. Those with ordinary skills in the art will appreciate that the elements in the figures are illustrated for simplicity and clarity and are not necessarily drawn to scale. There may be additional components or processes described in the foregoing application that are not depicted in the described drawings. In the event such a component or process is described, but not depicted in a drawing, the absence of such component and process from the drawings should not be considered as an omission of such design from the specification.

Before describing the present invention in detail, it should be observed that the present invention utilizes a combination of components or processes, which constitutes a method and system for building a family tree through the social network. Accordingly, the components or processes have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific component-level details and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

References to “one embodiment”, “an embodiment”, “another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment. The words “comprising”, “having”, “containing”, and “including”, and other forms thereof, are intended to be equivalent in meaning and be open ended in that an item or items following any one of these words is not meant to be an exhaustive listing of such item or items or meant to be limited to only the listed item or items. Further, the word “user” and “person” are used interchangeably in the description.

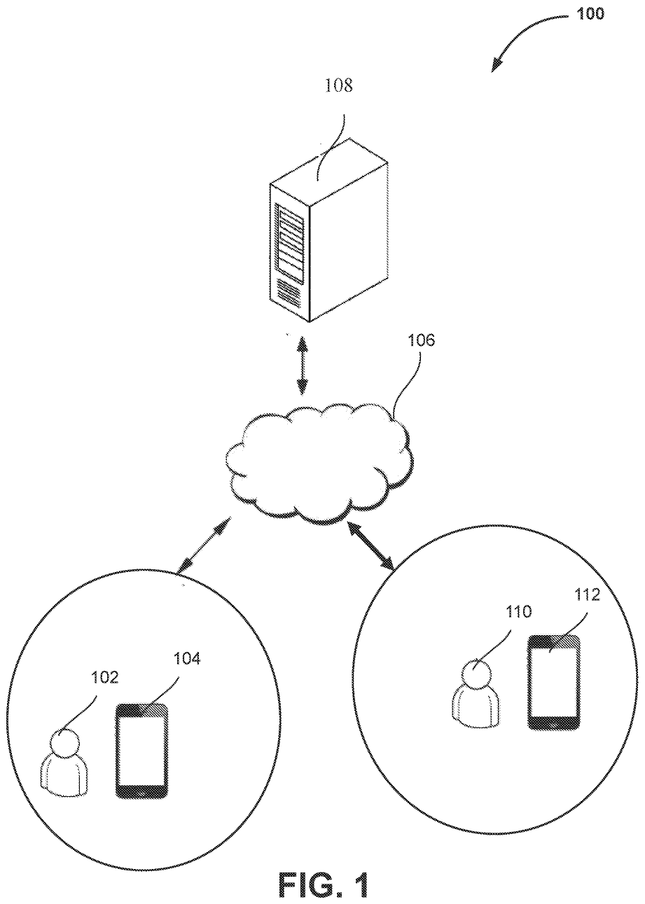

The system and method for building a family tree through a social network will now be described with reference to the accompanying drawings, particularly FIGS. 1-8.

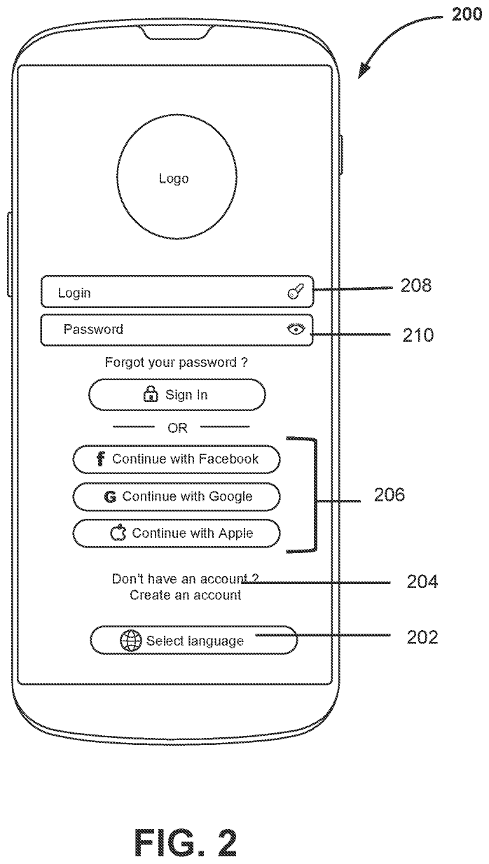

Referring to FIG. 1 in conjunction with FIGS. 2-8, a system 100 for building the family tree through the social network is shown, in accordance with an exemplary embodiment of the present invention. The system 100 includes a first person 102 who can access the social network that is hosted on a server 108 by providing login credentials. If the user is not a registered user of the social network, the user can register with the social network through the sign-up option. In an embodiment, the first person 102 can log in to the social network through the login credentials of another social network platform. In an embodiment, the server 108 is a cloud server. The first person 102 can access the social network through a computing device 104.

In the context of the present invention, the computing device 104 refers to an electronic device that can be used to communicate over the communication network 106. Examples of the computing device 104 include but are not limited to a cell phone, a smart phone, a cellular phone, a cellular mobile phone, a personal digital assistant (PDA), a personal computer, a server, a cloud-enabled devices, a laptop, and a tablet computer. Examples of types of the communication network 106 include, but are not limited to a local area network, a wide area network, a radio network, a virtual private network, an internet area network, a metropolitan area network, a satellite network, Wi-Fi, Bluetooth Low energy, a wireless network, and a telecommunication network. Examples of the telecommunication network include, but are not be limited to a global system for mobile communication (GSM) network, a general packet radio service (GPRS) network, third Generation Partnership Project (3GPP), 4G, Long-Term Evolution (LTE), an enhanced data GSM environment (EDGE) and a Universal Mobile Telecommunications System (UMTS).

In an embodiment, the first person 102 can access the social network through a client application related to the social network that is installed on the computing device 104. In another embodiment, the social network is a website hosted on the server 108, and the first person 102 can access the social network through a web browser that is installed on the computing device 104. In yet another embodiment, the social network is a browser add-on. In yet another embodiment, the social network is a cloud application.

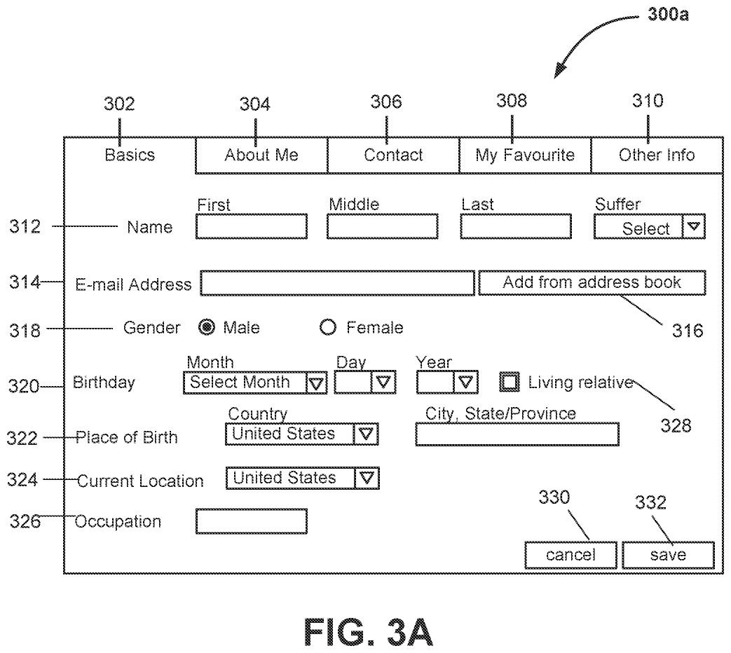

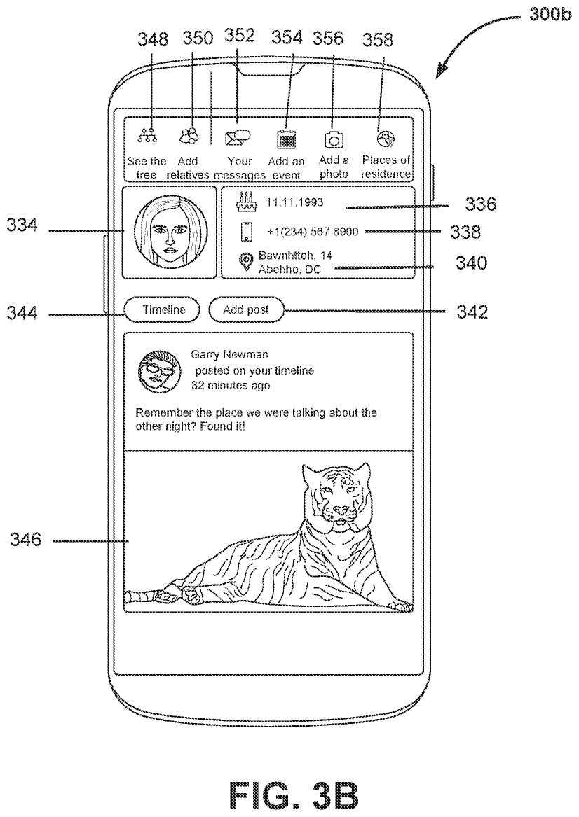

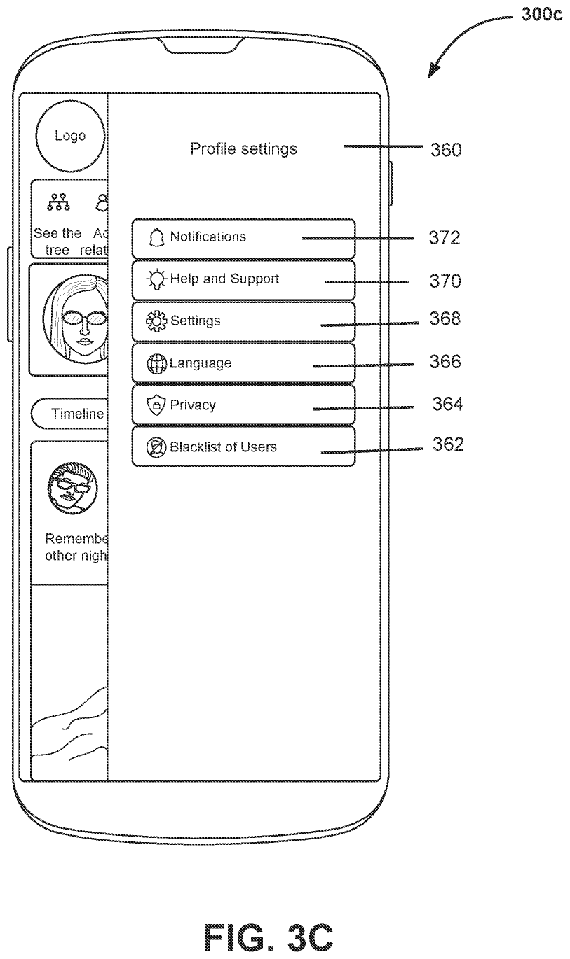

The first person 102 creates an account on the social network by providing sign-up information such as first name, last name, date of birth, email ID, and the like. After receiving the sign-up information from the first person 102, the social network creates a login credential for the first person 102 and shares the login credential with the first person 102 for subsequent login. The social network receives the login credentials from the first person 102 and provides access to a profile of the first person 102 in the social network based on the login credentials. An example user interface of the profile of the first person 102 is described in conjunction with FIG. 3B.

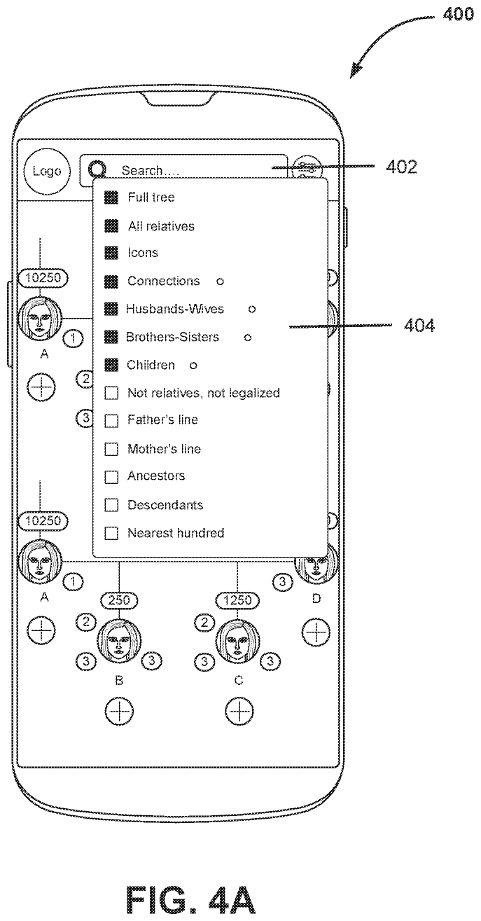

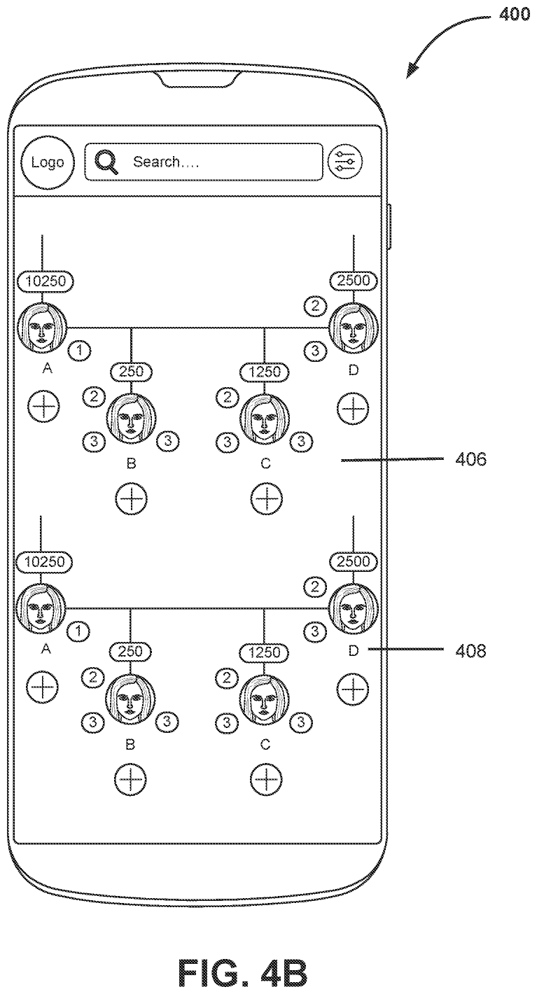

Further, the social network receives a first triggering event from the first person 102. In an embodiment, the first triggering event occurs when the first person 102 actuates the first web element in the profile of the first person 102. For example, the first person 102 clicks a web button 348 related to the family tree in the profile of the first person 102 as shown in FIG. 3B. In an embodiment, the web element includes one of a widget, a graphical control element, and a graphical user interface element. Further, the social network provides a user interface for building the family tree on receiving the first triggering event from the first person 102. The user interface for building the family tree allows the first person 102 to build his/her family tree from scratch. Further, the user interface for building the family tree allows the first person 102 to check whom the first person 102 can join or send invites to join the family tree. An example user interface for building the family tree is described in conjunction with FIGS. 4A-4B.

In an embodiment, the family tree is created by the linkage of a node to one or more additional nodes, each linkage having one or more than one defined linkage type. The linkage types include one of family relationship types, biological relationship types, and genetic relationship types. Further, one or more nodes of the family tree are displayed at a generation level. For example, Grandfather is in one generation level and father and uncle are in another generation level. The first person 102 can collapse or expand one or more of the predetermined generation levels to reveal additional generation levels. Further, each node is associated with information pertaining to the individual that it represents. Further, each of node provides an indication related to the current living status of a person; wherein the current living status comprises alive status or dead status. Further, each node provides indication related to the active user and inactive user; wherein an active user represents an individual who has registered with the social network and an inactive user represents an individual who has not registered with the social network.

Further, the social network receives a second triggering event from the first person 102. In an embodiment, the second triggering event occurs when the first person 102 actuates a second web element in the profile of the first person 102. In an example, the first person 102 clicks a save button on the user interface of the family tree to save changes related to the family tree. Further, the user interface of the family tree provides a search interface for filtering data based on one or more criteria; wherein one or more criteria comprises age, gender, geographical location, distance, current living status, date, time period, and the like. An example search interface for filtering data based on one or more criteria is described in conjunction with FIG. 4A. Further, the user interface of the family tree provides one or more web elements for zooming out and zooming in the family tree. In an embodiment, the social network includes one or more instant messaging, electronic mail, blogging, photo albums, calendar, and online diaries.

In an embodiment, the social network notifies users of the social network when information related to the user matches with the information of the first user; wherein the information related to the user includes relative names, birthdays, birthplaces, and contact information. Further, the social network allows users of the social network to send requests to each other to collaborate and join family trees.

Further, the social network determines whether a third triggering event from the first person 102 is received. In an embodiment, the third triggering event occurs when the first person 102 accepts an invitation from a second person 110. The second person 110 is another person that is registered with the social network. Further, the second person 110 may be a relative of the first person 102. The second person 110 is associated with a communication device 112. The second person 110 can access the social network through the communication device 112. In an embodiment, the first person 102 verifies the second person 110 based on data related to the invitation and whether the second person 110 is related to him/her and accepts the invitation. When the invitation is accepted, the social network updates the family tree automatically. Further, the social network displays an updated family tree on the user interface of the family tree. For example, if the second person 110 is the maternal uncle of the first person's mother and he sends an invitation to the first person 102 to add him to the family tree, the family tree will be updated automatically if the first person 102 accepts the invitation. If the first person 102 rejects the invitation, the family tree which was saved before receiving the invitation is displayed.

FIG. 2 illustrates an example login interface 200 for the social network, according to an embodiment of the present invention. The login interface 200 provides an option for the first person 102 to select the language for the social network. In an example, the first person 102 clicks a web button (shown as a globe icon) 202 to select the language for the social network. Further, the first person 102 can create an account on the social network by clicking the web button related to creating an account 204 and providing the required information. Furthermore, the first person 102 can log in to the social network through the login credentials of another social network platform. For example, the first person 102 selects any one of the network platforms from a list 206 to login into the social network. Additionally, the registered user, for example, the first person 102 can access the social network by providing login credentials in input boxes 208 and 210. The first person 102 provides a username in the first input box 208. Further, the first person 102 provides the password in the second input box 210.

FIG. 3A illustrates an example user interface 300a for entering data related to a first person 102, according to an embodiment of the present invention. According to one embodiment, there are tabs on the user interface 300a for entering data related to the first-person 102, and each tab is associated with a particular field. Basics tab 302 includes the fields 312 of the first name, middle name, last name, suffix, e-mail address(es) 314, gender 318, date of birth 320, living relative check box 328, place of birth 322, current location 324, and occupation 326. The first person 102 can click the save button 332 to save the entered data, otherwise the first person 102 can click the cancel button 330 to discard the changes. About tab 304 includes the fields of “about me” text-input area, nickname(s), alias(es), appearance, ethnicity, religion, orientation, political views, languages, and the like. In an example, the first person 102 enters a short description about himself/herself in the “about me” text-input area. Contact tab 306 includes home addresses, work addresses, phone numbers, and the like. My favourite 308 includes one's interests, activities, people/heroes, cuisines, quotes, movies, TV shows, music, books, sports, and the like. Other info 310 includes links to a web page, blog, other community sites, a diary, or any other links that are interested in the first person 102. The profile of the first person 102 is created based on the information collected from the user interface 300a. The first person 102 can access the profile after login into the social network.

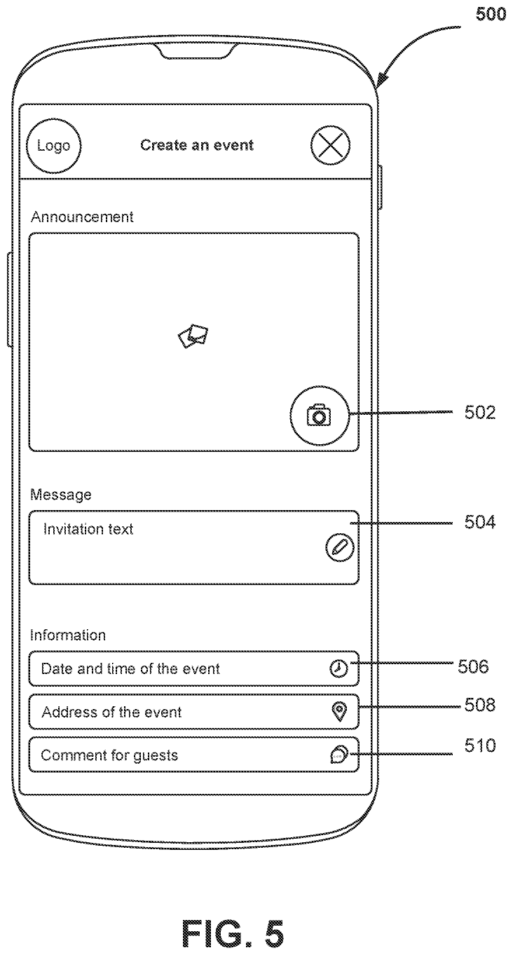

FIG. 3B illustrates an example user interface 300b of the user profile related to the first person 102, according to an embodiment of the present invention. According to one embodiment, there are tabs on the user interface 300b, and each tab is associated with a particular user interface. In an embodiment, the user interface 300b includes a tab 348 related to the family tree user interface. When the first person 102 clicks the tab 348, the user interface for building the family tree is provided. Further, the user interface 300b includes a tab 350 related to adding relatives to a list. For example, the list may be a family member list. In another example, the list may be a friend list. When the first person 102 clicks the tab 350, user interface for building a list is provided. Further, the user interface 300b includes a tab 352 related to messaging. The first person 102 can check the messages by clicking the tab 352. Further, the user interface 300b includes a tab 354 related to a user interface for adding an event. The first person 102 can create an event by clicking tab 354 and entering data related to the event. An example user interface for creating the event is described in conjunction with FIG. 5. Further, the user interface 300b includes a tab 356 related to adding a photo to an album of the first person 102 in the social network. The first person 102 can upload one or more images to the album by actuating the tab 356. Further, the user interface 300b includes a tab 358 related to residence address. The first person 102 can update his/her physical address.

Further, user interface 300b of the user profile includes information related to the first person 102 such as date of birth 336, phone number 338, and current address 340. Furthermore, a profile picture 334 of the first person 102 is displayed. The first person 102 can change the profile picture 334 by clicking the edit button related to the profile picture 334. Additionally, the first person 102 can check one or more post 346 on his/her timeline by clicking a “timeline” web button 344. Further, the first person 102 can add a post by clicking a “add post” web button 342 related to add a post.

FIG. 3C illustrates an example user interface 300c for applying settings 360 for the user profile, according to an embodiment of the present invention. To avoid unwanted interactions, the first person 102 can block a user of the social network by setting block action in “blacklist of users” tab 362. Further, the first person 102 can check a list of blocked users in the “blacklist of users” tab 362. Further, the first person 102 can apply and change the privacy setting in “Privacy” tab 364. Further, the first person 102 can select the language for the user profile by applying the language setting in “Language” tab 366. Further, the first person 102 can apply different settings to the user profile by enabling different options in “Settings” tab 368. Further, the first person 102 can connect to a technical team of the social network through “Help and Support” tab 370. Further, the first person 102 can apply to customize settings, for example, mute notification, for incoming notifications in “Notifications” tab 372.

FIG. 4A illustrates an example search interface 400 for filtering data based on one or more criteria 404, according to an embodiment of the present invention. The search interface 400 includes an input search box 402 where the first person 102 can enter text related to the criteria 404. Examples of the criteria 404 include, but are not limited to all relatives, icons, connections, Husbands-wives, Brothers-sisters, children, not relative, not legalized, father's line, mother's line, Ancestors, Descendants, Nearest hundred. The first person 102 can select the criteria 404 by selecting a check box against each of the criteria. When the first person 102 selects one of the criteria, for example, the father's line, the data related to the family tree is filtered out based on the father's line criterion. Similarly, the first person 102 can apply one or more criteria 404 to analyze the family tree.

FIG. 4B illustrates an example user interface 406 for displaying the filtered data based on one or more criteria 404, according to an embodiment of the present invention. When the first person 102 selects one of the criteria, for example, father's line, the data related to the family tree is filtered out based on the father's line criterion and is displayed on the user interface 406. In an embodiment, the family tree is created by the linkage of a node to one or more additional nodes, each linkage having one or more than one defined linkage type. Further, the linkage types include one of the familial relationship types, biological relationship types, and genetic relationship types. Further, one or more nodes of the family tree are displayed at a generation level. For example, Father is in one generation level and sons and daughters are in another generation level. The first person 102 can easily distinguish the generation level on the family tree visually. Further, each node is associated with information pertaining to the individual that it represents. Further, each the node provides an indication related to the current living status of a person; wherein the current living status comprises alive status or dead status. Further, each node provides indication related to active user and inactive user; wherein an active user represents an individual who has registered with the social network and an inactive user represents an individual who has not registered with the social network.

Further, each node is associated with a unique number which represents the relation of the person with his/her descendants. Further, the first person 102 can check the profile of an individual who is a part of the family tree by clicking the profile photo displayed on the node. In an embodiment, the user interface 406 provides web elements for zooming out and zooming in the family tree diagram. In an example, the first person 102 moves the slider of the web element to zoom out the family tree. In another example, the first person 102 clicks on the “+” sign on the web element to zoom out the family tree.

FIG. 5 illustrates an example user interface 500 for creating the event, according to an embodiment of the present invention. The first person 102 can add images to the event by clicking a camera icon 502. When the first person 102 clicks the camera icon 502, the user interface 500 provides an option to the user to upload an image. Further, the first person 102 can add text to the event through a text input box 504. Further, the first person 102 can add a date and time to the event by clicking a clock icon 506. When the first person 102 clicks the clock icon 506, a calendar is displayed on the user interface 500, and the first person 102 can select the date and time for the event. Further, the first person 102 can add an address to the event by clicking a map icon 508. When the first person 102 clicks the map icon 508, a map is displayed on the user interface 500, and the first person 102 can select a geographical location on the map for the event. Further, the first person 102 can add comments for guests who are coming to the event by clicking a chat icon 510. When the first person 102 clicks the chat icon 510, a text input box is displayed on the user interface 500 and the first person 102 can enter the comment into the text input box.

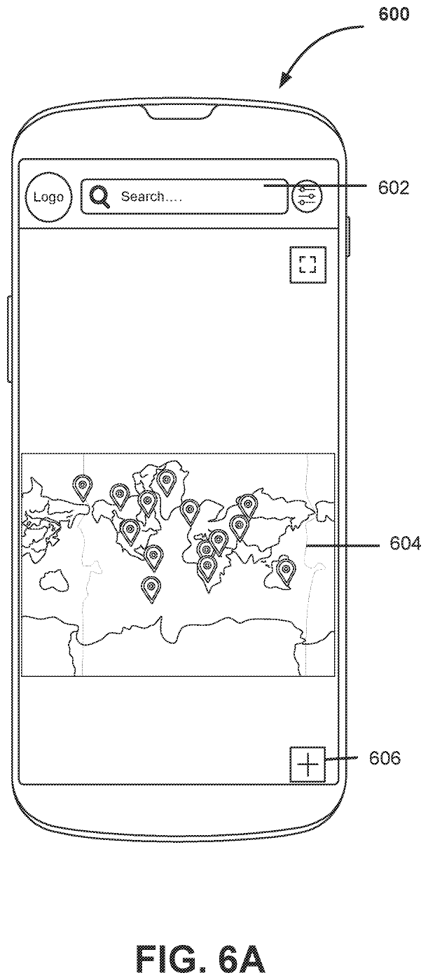

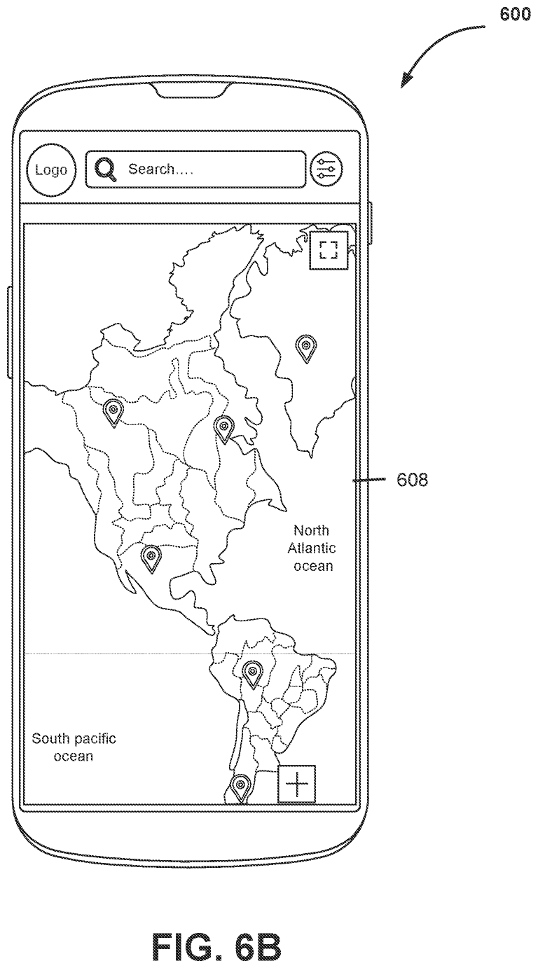

FIG. 6A illustrates an example user interface 604 for displaying zoom-in of the family tree diagram, according to an embodiment of the present invention. In an embodiment, the user interface 604 provides a web element 606 for zooming in the family tree diagram. In another example, the first person 102 clicks on a “−” sign on the web element to zoom in the family tree. In another example, the user interface 604 provides a pinch to zoom option for the family tree.

FIG. 6B illustrates an example user interface 608 for displaying zoom out of the family tree diagram, according to an embodiment of the present invention. In an embodiment, the user interface 604 provides the web element 606 to zoom out the family tree diagram. In another example, the first person 102 clicks on the “+” sign on the web element 606 to zoom in on the family tree. In another example, the user interface 608 provides pinch to zoom option to the family tree.

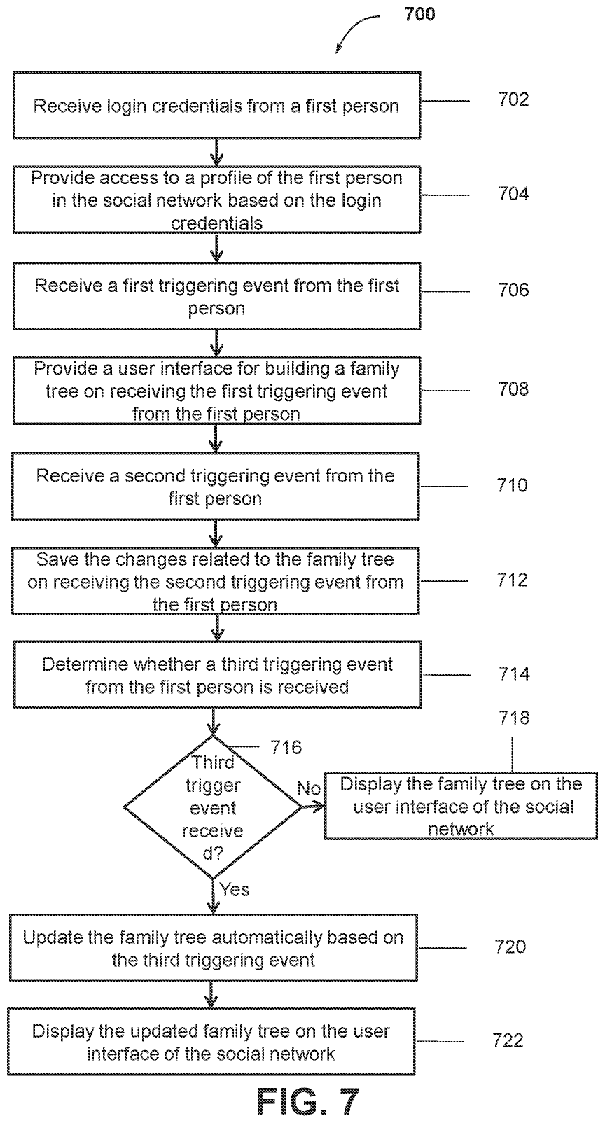

FIG. 7 illustrates a flow diagram 700 of a method for building the family tree through the social network, according to an exemplary embodiment of the present invention. At step 702, the method includes receiving the login credentials from the first person 102. The method allows the social network to receive the login credentials from the first person 102. At step 704, the method includes providing access to the profile of the first person 102 in the social network based on the login credentials. The method allows the social network to provide access to the profile of the first person 102 in the social network based on the login credentials. At step 706, the method includes receiving the first triggering event from the first person 102, wherein the first triggering event occurs when the first person 102 actuates the first web element in the profile of the first person. The method allows the social network to receive the first triggering event from the first person 102.

At step 708, the method includes providing the user interface for building the family tree on receiving the first triggering event from the first person 102. The method allows the social network to provide the user interface for building the family tree on receiving the first triggering event from the first person 102. In step 710, the method includes receiving the second triggering event from the first person 102, wherein the second triggering event occurs when the first person 102 actuates the second web element in the profile of the first person 102. The method allows the social network to receive the second triggering event from the first person 102. In step 712, the method includes saving the changes related to the family tree based on receiving the second triggering event from the first person 102. The method allows the social network to save the changes related to the family tree based on receiving the second triggering event from the first person 102. In step 714, the method includes determining whether the third triggering event from the first person 102 is received, wherein the third triggering event occurs when the first person 102 accepts an invitation from the second person 110. The method allows the social network to determine whether the third triggering event from the first person 102 is received.

If it is determined at step 716 that no third triggering event from the first person 102 is received, then the method at step 718 includes displaying the family tree on the user interface of the social network. The method allows the social network to display the family tree on the user interface of the social network. If it is determined at step 716 that the third triggering event from the first person 102 is received, then the method at step 720 includes updating the family tree automatically based on the third triggering event. The method allows the social network to update the family tree automatically based on the third triggering event. Further, in step 722, the method includes displaying the updated family tree on the user interface of the social network. The method allows the social network to display the updated family tree on the user interface of the social network.

The various actions acts, blocks, steps, or the like in the flow diagram may be performed in the order presented, in a different order, or simultaneously. Further, in some embodiments, some of the actions, acts, blocks, steps, or the like may be omitted, added, modified, skipped, or the like without departing from the scope of the invention.

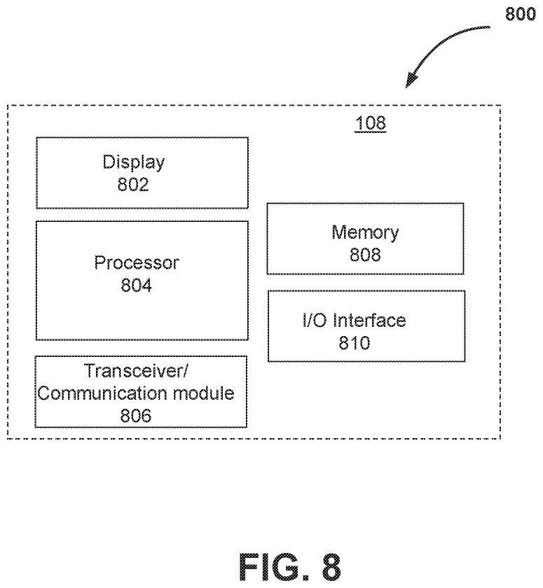

FIG. 8 illustrates a system 800 for building the family tree through the social network, according to an exemplary embodiment of the present invention. In accordance with an example implementation, as shown in FIG. 8, the system 800 may include at least a processor 804, a memory 808, a transceiver or communication module 806, and one or more I/O interfaces, such as an I/O interface 810, and a display 802.

The processor 804 may be communicably coupled with the transceiver/communication module 806 to receive data related to login credentials from the first person 102, and one or more triggering events from the first person 102. Further, the transceiver 806 may be configured to transmit data related to the family tree generated by the processor 804. The processor 804 is in communication with the memory 808, configured in the form of routines, programs, objects, components, data structures, and the like, which perform particular tasks to be executed by the processor 808. The system 800 may be connected to other information processing devices by using the I/O interface 810. The display 802 may be utilized to display one or more user interfaces as disclosed above. The I/O interfaces 810 may include a variety of software and hardware interfaces, for instance, interface for peripheral devices (s) such as a keyboard, a mouse, a scanner, an external memory, a printer, and the like.

In an embodiment, the processor 804 may include different types of processors known in the art including neural network-based algorithms that are effectively used in several applications.

The proposed invention provides many advantages over the existing methods and systems. The social network of the present invention helps people find who their ancestors are, who their relatives are, and further in the past they can build it with all family members input the more they could get informed about their family history and the more information could be passed on to generations to come as well as more living relatives would be found. Further, the social network of the present invention notifies the users if someone else who is using the social network of the present invention have matching relative names, birthdays, birthplaces, and contact information and allows people to send requests to each other to collaborate and join family trees, verify relatives of the combined family trees and see the relative contact information of the combined tree. Further, the system facilitates each member to start building their family tree from scratch and see whom they can join or send invites to family members via email or phone to join efforts of building their family tree.

Furthermore, the system facilitates the members of the social network platform to verify their family members or certain information or pictures of a certain member of the family or deny relationship to a certain member and also allow other members of the family to see whom certain member verified or denied and also how many family members verified certain member of the family on their family tree. The same type of verifications should be available for member contact information and pictures. Further, the social network of the present invention helps people find who their ancestors are, who their relatives are and further in the past they can build it with all family members' input the more they could get informed about their family history and the more information could be passed on to generations to come as well as more living relatives would be found.

Further, the social network of the present invention notifies the users if someone else who is using the social network platform of the present invention has matching relative names, birthdays, birthplaces, and contact information and allows people to send requests to each other to collaborate and join family trees, verify relatives of the combined family trees and see the relative contact information of the combined tree. One advantage of the present invention is that it helps each user of this invention more appreciate the value of a family Another advantage of the present invention is that it promotes good family values in society. One more advantage of the present invention is that it helps people find living relatives, as well as have a visual picture of how they are related. Yet another advantage of the present invention is that it helps people stay in contact with their living relatives as well as be informed of all events among their living relatives. Yet another advantage of the present invention is that it helps people see the geographical location of where their ancestors are from and where their living relatives live at present.

Although particular embodiments of the invention have been described in detail for purposes of illustration, various modifications and enhancements may be made without departing from the spirit and scope of the invention.

What is claimed is:

1. A method for building a family tree through a social network comprising:
receiving login credentials from a first person;
providing access to a profile of the first person in the social network based on the login credentials;
receiving a first triggering event from the first person; wherein the first triggering event occurs when the first person actuates a first web element in the profile of the first person;
providing a user interface for building a family tree on receiving the first triggering event from the first person;
receiving a second triggering event from the first person; wherein the second triggering event occurs when the first person actuates a second web element in the profile of the first person;
saving the changes related to the family tree on receiving the second triggering event from the first person;
determining whether a third triggering event from the first person is received; wherein the third triggering event occurs when the first person accepts an invitation from a second person;
displaying the family tree on the user interface of the social network based on the determination of receiving the third triggering event from the first person; wherein displaying the family tree further comprising:
displaying the family tree on the user interface of the social network if it is determined that no third triggering event from the first person is received; and
displaying an updated family tree on the user interface of the social network if it is determined that the third triggering event from the first person is received; wherein the displaying of the updated family tree further comprising:
updating the family tree automatically based on the third triggering event if it is determined that the third triggering event from the first person is received; and
displaying the updated family tree on the user interface of the social network.

2. The method of claim 1, wherein the family tree is created by the linkage of a node to one or more additional nodes, each linkage having one or more than one defined linkage type.

3. The method of claim 2, wherein the linkage types comprise one of familial relationship types, biological relationship types, and genetic relationship types.

4. The method of claim 2, further comprising displaying one or more nodes in a generation level.

5. The method of claim 4, further comprising collapsing or expanding one or more of a predetermined generation levels to reveal additional generation levels.

6. The method of claim 2, wherein each node is associated with information pertaining to the individual that it represents.

7. The method of claim 2, wherein each of the node provides indication related to the current living status of a person; wherein the current living status comprises alive status or dead status.

8. The method of claim 2, wherein each node provides indication related to active user and inactive user; wherein an active user represents an individual who has registered with the social network and an inactive user represents an individual who has not registered with the social network.

9. The method of claim 1, wherein web element comprises one of a widget, a graphical control element, and a graphical user interface elements.

10. The method of claim 1, further comprising providing a search interface for filtering data based on one or more criteria; wherein one or more criteria comprises age, gender, geographical location, distance, current living status, date, time period, and the like.

11. The method of claim 1, further comprising providing web elements for zoom out and zoom in the family tree diagram.

12. The method of claim 1, wherein the social network comprises one or more of instant messaging, electronic mail, blogging, photo-albums, calendar, and on-line diaries.

13. The method of claim 1, comprising notifying users of the social network when information related to the user matches with the information of the first user; wherein the information related to the user comprises relative names, birthdays, birthplaces, and contact information.

14. The method of claim 1, further comprising allowing users of the social network to send requests to each other to collaborate and join family trees.

15. The method of claim 1, accepting an invitation from a second person further comprising verifying the second person based on data related to the invitation.

16. A system for building a family tree through a social network comprising:
a memory;
a processor, coupled to the memory, configured to:
receive login credentials from a first person;
provide access to a profile of the first person in the social network based on the login credentials;
receive a first triggering event from the first person; wherein the first triggering event occurs when the first person actuates first web element in the profile of the first person;
provide a user interface for building a family tree on receiving the first triggering event from the first person;
receive a second triggering event from the first person; wherein the second triggering event occurs when the first person actuates second web element in the profile of the first person;
save the changes related to the family tree on receiving the second triggering event from the first person;
determine whether a third triggering event from the first person is received;
wherein the third triggering event occurs when the first person accepts an invitation from a second person;
display the family tree on the user interface of the social network based on the determination of receiving the third triggering event from the first person; wherein displaying the family tree further comprising:
display the family tree on the user interface of the social network if it is determined that no third triggering event from the first person is received; and
display an updated family tree on the user interface of the social network if it is determined that the third triggering event from the first person is received; wherein the displaying of the updated family tree further comprising:
update the family tree automatically based on the third triggering event if it is determined that the third triggering event from the first person is received; and
displaying the updated family tree on the user interface of the social network.

17. The system of claim 16, wherein the processor further configured to verify the second person based on data related to the invitation.

18. A non-transitory computer-readable medium having stored thereon a plurality of instructions, the plurality of instructions when executed by a computer, cause the computer to perform:
receiving login credentials from a first person;
providing access to a profile of the first person in the social network based on the login credentials;
receiving a first triggering event from the first person; wherein the first triggering event occurs when the first person actuates first web element in the profile of the first person;
providing a user interface for building a family tree on receiving the first triggering event from the first person;
receiving a second triggering event from the first person; wherein the second triggering event occurs when the first person actuates second web element in the profile of the first person;
saving the changes related to the family tree on receiving the second triggering event from the first person;
determining whether a third triggering event from the first person is received; wherein the third triggering event occurs when the first person accepts an invitation from a second person;
displaying the family tree on the user interface of the social network based on the determination of receiving the third triggering event from the first person; wherein displaying the family tree further comprising:
displaying the family tree on the user interface of the social network if it is determined that no third triggering event from the first person is received; and
displaying an updated family tree on the user interface of the social network if it is determined that the third triggering event from the first person is received; wherein the displaying of the updated family tree further comprising:
updating the family tree automatically based on the third triggering event if it is determined that the third triggering event from the first person is received; and
displaying the updated family tree on the user interface of the social network.
